SEQUENCE LISTING (15 sequences)

SEQ ID NO: 1 - protein, 462 aa, Artificial Sequence
  Humanised sequence of mus musculus and homo sapiens
MGWSCIILFLVATATGVHSQVQLVQSGAEVKKPGASVKVSCKASGYTFTSYWMHWVRQAPGQGLEWIGNINPSNGGTNYNEKFKSKATMTRDTSTSTAYMELSSLRSEDTAVYYCELMQGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 238 aa, Artificial Sequence
  Humanised sequence of mus musculus and homo sapiens
MGWSCIILFLVATATGVHSDIVMTQSPLSNPVTLGQPVSISCRSSKSLLYKDGKTYLNWFLQRPGQSPQLLIYLMSTRASGVPDRFSGGGSGTDFTLKISRVEAEDVGVYYCQQLVEYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 1389 bp, Artificial Sequence
  Humanised sequence of mus musculus and homo sapiens
atgggatggagctgtatcatcctcttcttggtagcaacagctacaggtgtccactcccaggtgcagctggtgcagtctggggctgaggtgaagaagcctggggcctcagtgaaggtttcctgcaaggcatctggatacaccttcaccagctactggatgcactgggtgcgacaggcccctggacaagggcttgagtggatcggaaatattaatcctagcaatggtggtactaactacaatgagaagttcaagagcaaggccaccatgaccagggacacgtccacgagcacagcctacatggagctgagcagcctgagatctgaggacacggccgtgtattactgtgaactgatgcagggctactggggccagggaacactagtcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 4 - DNA, 717 bp, Artificial Sequence
  Humanised sequence of mus musculus and homo sapiens
atgggatggagctgtatcatcctcttcttggtagcaacagctacaggtgtccactccgatattgtgatgacccagtctccactctccaaccccgtcacccttggacagccggtctccatctcctgcaggtctagtaagagtctcctatataaggatgggaagacatacttgaattggtttctccagaggccaggccaatctccacagctcctaatttatttgatgtccacccgtgcatctggggtcccagacagattcagcggcggtgggtcaggcactgatttcacactgaaaatcagcagggtggaggctgaggatgttggggtttattactgccaacaacttgtagagtatccgctcacgtttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggacaacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 5 - protein, 445 aa, Artificial Sequence
  Humanised sequence of mus musculus and homo sapiens
EVQLVESGGGLVQPGGSLRLSCAVSGFTFSDNGMAWVRQAPGKGLEWVSFISNLAYSIDYADTVTGRFTISRDNAKNSLYLQMNSLRAEDTAVYYCVSGTWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 219 aa, Artificial Sequence
  Humanised sequence of mus musculus and homo sapiens
DIVMTQSPLSLPVTPGEPASISCRVSQSLLHSNGYTYLHWYLQKPGQSPQLLIYKVSNRFSGVPDRFSGSGSGTDFTLKISRVEAEDVGVYYCSQTRHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 442 aa, Artificial Sequence
  Humanised sequence of mus musculus and homo sapiens
QVQLVQSGAEVKEPGASVKVSCKGSGFNIKVYYVHWLRQLPGKGLEWIGRIDPENGETIYTPKFQDKATLTVDTSTDTAYMELSSLRSEDTAVYYCVSSGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 218 aa, Artificial Sequence
  Humanised sequence of mus musculus and homo sapiens
DIVMTQSPLSNPVTPGEPASISCRSSKSLLHRNGITYLYWYLQKPGQSPQLLIYQMSNLASGVPDRFSSSGSGTDFTLKISRVEAEDVGVYYCAQNLELWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 6 aa, Artificial Sequence
  Humanised sequence of mus musculus and homo sapiens
YAGFLR

SEQ ID NO: 10 - protein, 130 aa, Artificial Sequence
  Humanised sequence of mus musculus and homo sapiens
EVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSSAAAEQKLISEEDLN

SEQ ID NO: 11 - protein, 115 aa, Artificial Sequence
  Humanised sequence of mus musculus and homo sapiens
DIVMTQSPDSLAVSLGERATINCKSSHSVLYSSNQKNYLAWYQQKPGQPPKLLIYWASTRESGVPDRFSGSGSGTDFTLTISSLQAEDVAVYYCHQYLSSLTFGQGTKLEIKRTV

SEQ ID NO: 12 - protein, 126 aa, Artificial Sequence
  Humanised sequence of mus musculus and homo sapiens
QVQLVQSGSELKKPGASVKVSCKASGYTFTNYGMNWVRQAPGQGLEWMGWINTYTGEPTYADDFTGRFVFSLDTSVSTAYLQISSLKAEDTAVYYCARNPINYYGINYEGYVMDYWGQGTLVTVSS

SEQ ID NO: 13 - DNA, 1335 bp, Artificial Sequence
  Humanised sequence of mus musculus and homo sapiens
gaggtgcagctggtggagtctgggggaggcttggtacagcctggggggtccctgagactctcctgtgcagtctctggattcaccttcagtgacaacggaatggcgtgggtccgccaggctccagggaaggggctggagtgggtttcattcattagtaatttggcatatagtatcgactacgcagacactgtgacgggccgattcaccatctccagagacaatgccaagaactcactgtatctgcaaatgaacagcctgagagccgaggacacggctgtgtattactgtgtcagcgggacctggtttgcttactggggccagggcacactagtcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 14 - DNA, 657 bp, Artificial Sequence
  Humanised sequence of mus musculus and homo sapiens
gacatcgtgatgacccagagccccctgagcctgcccgtgacccctggcgagcccgccagcatcagctgtagagtgagccagagcctgctgcacagcaacggctacacctacctgcactggtatctgcagaagcctggccagagccctcagctgctgatctacaaggtgtccaaccggttcagcggcgtgcctgatagattcagcggcagcggctccggcaccgacttcaccctgaagatcagcagagtggaggccgaggatgtgggcgtgtactactgctcccagaccagacacgtgccttacacctttggcggcggaacaaaggtggagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 15 - DNA, 657 bp, Artificial Sequence
  Humanised sequence of mus musculus and homo sapiens
gatattgtgatgactcagtctccactctccctgcccgtcacccctggagagccggcctccatctcctgcagagttagtcagagccttttacacagtaatggatacacctatttacattggtacctgcagaagccagggcagtctccacagctcctgatctataaagtttccaaccgattttctggggtccctgacaggttcagtggcagtggatcaggcacagattttacactgaaaatcagcagagtggaggctgaggatgttggggtttattactgctctcaaactagacatgttccgtacacgttcggcggagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggacaacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

(19) United States
(12) Patent Application Publication
(10) Pub. No.: US 2011/0064821 A1
(43) Pub. Date: Mar. 17, 2011

(54) ENCAPSULATION OF BIOLOGICALLY ACTIVE AGENTS

(76) Inventors: Ian Richard Catchpole, Hertfordshire (GB); Gerald Wayne Gough, Hertfordshire (GB); Irene Papanicolaou, Hertfordshire (GB)

(21) Appl. No.: 12/991,556

(22) PCT Filed: May 5, 2009
(86) PCT No.: PCT/EP2009/055436

§ 371 Date: Nov. 8, 2010

Related U.S. Application Data
(60) Provisional application No. 61/050,775, filed on May 6, 2008.
(60) Provisional application No. 61/074,171, filed on Jun. 20, 2008.

(51) Int. Cl. A61K 9/14; A61K 38/16; A61K 39/395; A61P 27/02; B82Y 5/00

(52) U.S. Cl. 424/501; 514/20.8; 424/130.1; 977/773

(57) ABSTRACT

The present invention provides methods of encapsulating biologically active agents such as proteins in particulate carriers such as nanoparticles. Also provided are methods of delivery of proteins across the blood brain barrier in nanoparticles, and uses of such compositions in treatment.

### BACKGROUND

A number of drugs have activity at targets in the brain, in order to get these to their target they must pass through the blood brain barrier. While some molecules are able to cross biological barriers, there are others which do not pass these barriers efficiently or in fact at all. Many drugs are also only efficient when given directly into the target tissue and if this target tissue cannot be reached the drug simply cannot work. Therefore many potentially potent drugs are not useful clinically due to their inability to pass such biological barriers.

A number of approaches have been described in the art to increase drug penetration through these biological barriers.

One approach has been to alter the function of the barrier itself. For instance, osmotic agents or cholinomimetic arecolines result in the opening, or a change in the permeability, of the blood brain barrier (Saija A et al, J Pharm. Pha. 42:135-138 (1990)).

Another approach resides in the modification of the drug molecules themselves. For instance modifications of proteins to attempt passage across the blood brain barrier include glycating such proteins, or alternatively by forming a prodrug. (WO/2006/029845).

Still another approach is the implantation of controlled release polymers which release the active ingredient from a matrix system directly into the nervous tissue. However, this approach is invasive and requires surgical intervention if implanted directly into the brain or spinal cord (sable et al. U.S. Pat. No. 4,833,666) this presents problems with patient compliance and often only allows for localised delivery within the brain with the administered drug usually draining away very quickly. (WO/2006/029845).

To overcome these limitations drug carrier systems have been used however, a major problem in targeted drug delivery is the rapid opsonisation and uptake of injected carriers by the reticuloendothelial system (RES) especially by the macrophages in the liver and spleen.

There remains therefore a need for an efficient and effective means of delivering macromolecules such as proteins to the brain. In particular, it would be desirable to find a method of delivery of macromolecules across the blood brain barrier, which would retain activity on entry into the brain, and which may also provide desirable release kinetics, maintain protein stability and activity, and have the ability to evade clearance mechanisms.

### BRIEF DESCRIPTION OF THE FIGURES

FIG. 1. FIG. 1 shows the sizing data obtained by DLS that indicate the presence of nanoparticles in suspension.

FIG. 1(a) Correlogram obtained following analysis of a nanoparticle suspension by dynamic light scattering. According to the data obtained, the mean hydrodynamic diameter of the particles was 292.5 nm and the polydispersity index 0.250.

FIG. 1(b) Multimodal size distribution of the nanoparticles plotted to depict the distribution of the particle population (number) over a range of sizes. The majority of the particle population appears to have a diameter of around 40 nm.

FIG. 1(c) Multimodal size distribution (derived data) of the nanoparticles plotted to depict the distribution of the particle population (number) over a range of sizes. The data suggest that 87.5% of the particle sample possesses a diameter of 138.19 nm or lower.

FIG. 1(d) Multimodal size distribution (derived data) of the nanoparticles plotted to depict the distribution of the particle population (number) over a range of sizes. The data suggest that 14.9% of the particle sample possesses a diameter of 99.86 nm or lower.

FIG. 2.—Nanoparticles analysed by SEM

FIG. 3—Image of hollow nanoparticles by TEM, with a superimposed image of solid PBCA nanoparticles for comparison.

FIG. 4—Encapsulation efficiency measurements of monoclonal IgG1 (anti-CD23). The encapsulation efficiency was found to be 52% when using 600 μg of antibody.

FIG. 5—Release profile obtained following enzymatic degradation of particles and analysis of the released enzyme by ELISA.

FIG. 6—Determination of the encapsulation efficiency of a domain antibody (hen egg lysozyme dAb) by the bicinchoninic acid assay (BCA assay) in a hollow PBCA nanoparticle. The encapsulation efficiency was found to be 66%. The loading efficiency was 11.1%.

FIG. 7. Sizing data obtained by DLS that indicate the presence of nanoparticles in suspension.

FIG. 7(a)—Correlogram obtained following analysis of a nanoparticle suspension by dynamic light scattering. According to the data obtained, the mean hydrodynamic diameter of the particles was 291.4 nm and the polydispersity index 0.242.

FIG. 7(b)—Multimodal size distribution of the nanoparticles plotted to depict the distribution of the particle population (number) over a range of sizes. The majority of the particle population appears to have a diameter of around 164 nm.

FIG. 7(c)—Multimodal size distribution (derived data) of the nanoparticles plotted to depict the distribution of the particle population (number) over a range of sizes. The data suggest that 96.4% of the particle sample possesses a diameter of 201.37 nm or lower.

FIG. 7(d)—Multimodal size distribution (derived data) of the nanoparticles plotted to depict the distribution of the particle population (number) over a range of sizes. The data suggest that 6.2% of the particle sample possesses a diameter of 143.38 nm or lower.

FIG. 8. Comparison of the amounts of encapsulated Dalargin achieved with the HIP to those achieved by the common method of adsorption onto the particle surface clearly demonstrated the superiority of the HIP-PBCA process.

FIG. 9—Dalargin levels in the brain following delivery with HIP-PBCA nanoparticles. The peptide was detectable in the brain only when encapsulated within the particles using the HIP process. The highest brain concentration, at 45 ng/ml, was achieved with the 5:1 HIP-PBCA formulation. No peptide was detectable in the brain when it was given free in solution or when it was adsorbed onto the nanoparticles.

FIG. 10.—Encapsulation of dalargin into PBCA nanoparticles using the HIP process. Determination of the effect if pH of the aqueous phase on the encapsulation efficiency. The encapsulation efficiency was higher when the particles were formed at pH7, than when they were prepared at pH 2. At a HIP:dalargin ratio of 10:1, 63.23% of the input peptide was entrapped into the nanoparticles when the particles were formed at pH 7. At pH 2, the encapsulation efficiency was significantly lower at 2.36%.

FIG. 11.—Encapsulation of anti-hen egg lysozyme domain antibody into PBCA nanoparticles using the HIP process. The nanoparticles were analysed by Edman sequencing. The first repeat of the formulation (repeat 1) gave a total of 2.56 mg encapsulated peptide. The second repeat (repeat 2) gave a lower result at 1.72 mg, probably due to loss of part of the sample during the analysis.

FIG. 12—Encapsulation efficiency measurements of monoclonal IgG1 (anti-CD23). The encapsulation efficiency was found to be 86.2% when using 8.6 mg of antibody.

### SUMMARY OF INVENTION

In one aspect of the present invention there is provided a method of encapsulating biologically active agents in particulate carriers such a methods of encapsulating proteins in nanoparticles and a method of delivery of proteins across the blood brain barrier by encapsulation in nanoparticles.

In another embodiment of the present invention there are provided nanoparticles comprising a particle forming substance and a protein, for delivery of a protein from the blood to the brain across the blood brain barrier. In another embodiment of the invention are compositions of nanoparticles and their use in treating disorders or diseases of the central nervous system.

### DETAILED DESCRIPTION OF INVENTION

The present invention provides nanoparticles comprising a particle forming substance and a protein, for delivery of the protein from the blood to the brain across the blood brain barrier.

In one embodiment the nanoparticles of the present invention comprise antigen binding molecules, such molecules may comprise at least one immunoglobulin variable domain, for example such molecules may comprise an antibody, a domain antibody, Fab, Fab′, F(ab′)2, Fv, ScFv, diabody, mAb-dAb, affibody, heteroconjugate antibody. Such antigen binding molecules may be capable of binding to a single target, or may be multispecific, i.e. bind to a number of targets, for example bispecific or trispecific. In one embodiment the antigen binding molecule is an antibody. In another embodiment the antigen binding molecule is a dAb. In yet a further embodiment the antigen binding molecule may be a combination of antibodies and antigen binding fragments such as for example, one or more domain antibodies and or ScFv attached to a monoclonal antibody.

In one embodiment of the invention the protein binds to a target found in the central nervous system such as for example in the brain or spinal cord, or for example in neuronal tissue.

In yet a further embodiment of the invention described herein the protein specifically binds to an epitope known to be linked to neurological diseases or disorders such as for example MAG (myelin associated glycoprotein), NOGO or β-amyloid.

In one embodiment of the present invention there is provided a composition comprising nanoparticles of the invention. In a further embodiment at least about 90% of the nanoparticles by number are within the range of about 1 nm to about 1000 nm when measured using dynamic light scattering techniques. In a further embodiment at least about 90% of the nanoparticles by number are within the range of about 1 nm to about 400 nm, or about 1 nm to about 250 nm or about 1 nm to about 150 nm, or about 40 nm to about 250 nm, or about 40 nm to about 150 nm, or about 40 nm to about 100 nm when measured using dynamic light scattering techniques.

In yet a further embodiment of the present invention at least about 90% of the nanoparticles by number are within the range of about 40 nm to about 250 nm when measured using dynamic light scattering techniques.

In yet a further embodiment of the present invention at least about 90% of the nanoparticles by number are within the range of about 40 nm to about 150 nm when measured using dynamic light scattering techniques.

In yet a further embodiment there is provided a composition comprising the nanoparticles of the present invention wherein the median size of the nanoparticles in the composition is less than about 1000 nm in diameter, for example is less than about 400 nm in diameter for example is less than about 250 nm in diameter, for example is less than about 150 nm in diameter when measured by light scattering techniques.

In yet a further embodiment the median size of the nanoparticles in the composition is about 40 nm to about 250 nm.

In yet a further embodiment the median size of the nanoparticles in the composition is about 40 nm to about 150 nm.

In one embodiment of the present invention there is provided a method of encapsulating biologically active agents in a particulate carrier comprising the steps of:
a) solubilising a biologically active agent in the presence of a hydrophobic ion pairing (HIP) agent and in an organic solvent;
b) dissolving a monomer of a polymer forming substance in the organic phase formed in (a);
c) forming an emulsion of the organic phase formed in (b) in a continuous aqueous phase to allow polymerisation of the monomer; and
d) obtaining particulate carriers formed from the emulsion.

This method using hydrophobic ion pairing agents allows encapsulation of biologically active agents for example proteins such as for example hydrophilic proteins within the core of the hydrophobic polymer particles. The method described herein achieves encapsulatedbiologically active agents throughout the particle volume. Hydrophobic ion pairing allows extraction of protein into an organic medium and therefore the method enables preparation of a particulate carrier with a single emulsion.

In a further embodiment the biologically active agents to be encapsulated in accordance with the method are peptides or proteins, such as therapeutic proteins or antigen binding molecules.

The antigen binding molecule of the present invention may be a molecule comprising at least one Ig variable domain, for example an antibody, domain antibody, Fab, Fab′, F(ab′)2, Fv, ScFv, diabody, mAb-dAb, affibody, heteroconjugate antibody. Such antigen binding molecules may be capable of binding to a single target, or may be multispecific, i.e. bind to a number of targets, for example bispecific or trispecfic. In one embodiment the antigen binding molecule is an antibody. In another embodiment the antigen binding molecule is a dAb. In yet a further embodiment the antigen binding molecule may be a combination of antibodies and antigen binding fragments such as for example, one or more domain antibodies and or ScFv attached to a monoclonal antibody. In yet a further embodiment the antigen binding molecule may be a combination of antibodies and antigen binding fragments such as for example, one or more domain antibodies and or ScFv bound to a monoclonal antibody.

In one embodiment of the invention the protein binds to a target found in the central nervous system such as for example in the brain or spinal cord or for example in the neuronal tissue.

In a further embodiment the particulate carriers may be microspheres or nanoparticles. In a further embodiment the particulate carrier is a nanoparticle and the biologically active agent is a protein. In a further embodiment the particulate carrier is a nanoparticle and the biologically active agent is a peptide. In yet a further embodiment the particulate carrier is a nanoparticle and the biologically active agent comprises an antigen binding molecule for example a domain antibody or antibody. In another embodiment the particulate carrier is a microsphere and the biologically active agent is a protein. In a further embodiment the particulate carrier is a microsphere and the biologically active agent is a peptide. In yet a further embodiment the particulate carrier is a nanoparticle and the biologically active agent comprises an antigen binding molecule for example a domain antibody or antibody In one embodiment of the invention described herein the biologically active agent specifically binds to an epitope known to be linked to neurological diseases or disorders such as for example to MAG, NOGO, or β-amyloid.

In one embodiment the biologically active agent is insoluble in the organic phase without the presence of hydrophobic ion pairing agents.

In one embodiment of the invention as herein described the hydrophobic ion pairing agent is a cationic HIP agent when the protein is anionic. In another embodiment the hydrophobic ion pairing agent is an anionic HIP agent when the protein is cationic. In a further embodiment the anionic HIP agent is selected from the group consisting of Alkyl quaternary ammonium cations, preferably alkyl ammonium bromides, more preferably tetrabutyl ammonium bromide, tetrahexyl ammonium bromide, tetraoctyl ammonium bromide, Sodium dodecyl sulphate (SDS), sodium oleate or docusate sodium (aka Aerosol OT) and the HIP agent is present in stoichiometric amounts equal to or greater than the number of net positive charges on the protein. In another embodiment, the cationic HIP agent is selected from the group consisting of: dimethyldioctadecyl-ammonium bromide (DDAB18); 1,2-dioleoxy-3-(trimethylammonium propane (DOTAP); or cetrimonium bromide (CTAB) and the HIP agent is present in stoichiometric amounts equal to or greater than the number of net negative charges on the protein.

In a further embodiment any hydrophobic cation or anion could potentially be used as a HIP agent. Hydrophobic ion pairing (HIP) involves stoichiometric replacement of polar counter ions with a species of similar charge but less easily solvated. As disclosed herein, the invention provides a method that uses HIP to change the solubility properties of proteins, allowing extraction of the protein into an organic solvent, such as methylene chloride. Ducosate sodium (Bis(2-ethylhexyl) sodium sulfosuccinate) is one example of a suitable ion-pairing agent. In one embodiment, methylene chloride containing ducosate sodium is mixed with an aqueous protein solution. This results in ion pairing of the ducosate ion with the protein and subsequent partitioning of the protein into the oil phase. Dissolution of the protein in methylene chloride allows the protein to be encapsulated in nanoparticles or microspheres prepared via a single oil-in-water emulsion method.

In one embodiment of the invention herein described the continuous aqueous phase has a pH of about 7.0 or higher when the protein is anionic and the HIP agent is cationic, for example the pH may be at least about 8.0 or at least about 10.0 or is at least about 12.0.

In an alternative embodiment of the invention herein described the continuous aqueous phase has a pH of about 7.0 or lower when the protein is cationic and the HIP agent is anionic, for example the pH may be less than about 6.0 or less than about 4.0 or less than about 2.0.

In one such embodiment the W/W ratio of protein to polymer may be 0.5% to 50% for example is at least about 0.5% or is at least about 1% or is at least about 2% or is at least about 2.5% or is at least about 5% or is at least about 9% or is at least about 10% or is at least about 15% or is at least about 20% or is at least about 40%, or is at least about 50%. For example when the protein is a peptide the peptide to polymer ratio may be at least about 9%, when the protein is an antibody the antibody to polymer ratio may be at least about 2%, or when the protein is a domain antibody domain antibody to polymer ratio may be at least about 2.5%.

In one embodiment of the present invention the W/W ratio of protein to total formulation may be 0.5% to 50% for example is at least about 5% or at least about 9% or at least about 15% or at least about 16% or at least about 20% or at least about 25%. For example when the protein is a peptide the peptide to total formulation ratio may be at least about 16% or when the protein is a domain antibody the domain antibody to total formulation ratio may be at least about 9%.

In one embodiment of the present invention the monomer is selected from the group consisting of: methylmethacrylates, alkylcyanoacrylates, hydroxyethylmethacrylates, methacrylic acid, ethylene glycol dimethacrylate, acrylamide, N,N′-bismethylene acrylamide and 2-dimethylaminoethyl methacrylate.

In a further embodiment the monomer is an alkylcyanoacrylate for example is butylcyanoacrylate (BCA).

In one embodiment of the present invention there is provided a method of encapsulating biologically active agents in particulate carriers for ocular delivery comprising the steps of:
a) dissolving a polymer in an organic solvent to form a polymer solution;
b) adding an aqueous solution containing a biologically active agent to the polymer solution to form a primary emulsion of aqueous phase droplets in a continuous organic phase;
c) mixing the primary emulsion with an aqueous medium to form a secondary emulsion; and
d) allowing the organic phase to evaporate and thereby obtain particulate carriers comprising a hollow lumen containing biologically active agents in an aqueous phase.

In one such embodiment the ocular delivery is periocular, for example trans-scleral, subconjunctival, sub-tenon, peribulbar, topical, retrobulbar or is delivered to the inferior, superior or lateral rectus muscle. In one embodiment the ocular delivery is trans-scleral.

Such biologically active molecules may be a peptide or protein, such as a therapeutic protein or antigen binding molecule for example an antibody or a domain antibody or a combination thereof.

For the avoidance of doubt the term “Biologically active agent” and the “biologically active molecule” as used throughout the specification are intended as to have the same meaning and able to be used interchangeably.

the particulate carrier used in such methods may be a microsphere or a nanoparticle For example the particulate carrier may be a nanoparticle and the biologically active agent a protein or the particulate carrier may be a nanoparticle and the biologically active agent a peptide or the particulate carrier may be a nanoparticle and the biologically active agent comprises an antigen binding molecule such as a domain antibody or antibody.

Alternatively the particulate carrier may be a microsphere and the biologically active agent a protein or the particulate carrier may be a microsphere and the biologically active agent a peptide or the particulate carrier may be a microsphere and the biologically active agent comprises an antigen binding molecule such as a domain antibody or antibody or a combination thereof.

In one embodiment at least about 90% of the microspheres by number are within the range of about 1 μm to about 100 μm when measured using Low angle laser light scattering techniques.

In a further embodiment at least about 90% of the microspheres by number are within the range of about 1 μm to about 80 μm, or about 1 μm to about 60 μm or about 1 μm to about 40 μm, or about 1 μm to about 30 μm or about 1 μm to about 10 μm when measured using Low angle laser light scattering techniques.

In yet a further embodiment of the present invention at least about 90% of the microspheres by number are within the range of about 1 μm to about 60 μm when measured using Low angle laser light scattering techniques.

In yet a further embodiment of the present invention at least about 90% of the microspheres by number are within the range of about 1 μm to about 30 μm when measured using Low angle laser light scattering techniques.

In yet a further embodiment there is provided a composition comprising the microspheres of the present invention wherein the median size of the microspheres in the composition is less than about 100 μm in diameter, for example is less than about 80 μm in diameter for example is less than about 60 μm in diameter, for example is less than about 40 μm in diameter when measured by Low angle laser light scattering techniques.

In yet a further embodiment the median size of the microspheres in the composition is about 1 μm to about 6 μm, or 1 μm to about 30 μm.

In one embodiment of the present invention there is provided a method of producing the nanoparticles of the invention as herein described comprising the steps of:
a) dissolving a polymer in an organic solvent to form a polymer solution;
b) adding an aqueous solution containing protein to the polymer solution to form a primary emulsion of aqueous phase droplets in a continuous organic phase;
c) mixing the primary emulsion with an aqueous medium to form a secondary emulsion; and
d) allowing the organic phase to evaporate and thereby obtain nanoparticles comprising a hollow lumen containing proteins in an aqueous phase.

In a further embodiment the polymer used in any of the methods as described herein is selected from but not limited to: poly-L-lactide (PLA), poly(lacto-co-glycolide) (PLG), poly(lactide), poly(caprolactone), poly(hydroxybutyrate) and/or copolymers thereof. Suitable particle-forming materials include, but are not limited to, poly(dienes) such as poly(butadiene) and the like; poly(alkenes) such as polyethylene, polypropylene, and the like; poly(acrylics) such as poly(acrylic acid) and the like; poly(methacrylics) such as poly(methyl methacrylate), poly(hydroxyethyl methacrylate), and the like; poly(vinyl ethers); poly(vinyl alcohols); poly(vinyl ketones); poly(vinylhalides) such as poly(vinyl chloride) and the like; poly(vinyl nitriles), poly(vinyl esters) such as poly(vinyl acetate) and the like; poly(vinyl pyridines) such as poly(2-vinyl pyridine), poly(5-methyl-2-vinyl pyridine) and the like; poly(styrenes); poly(carbonates); poly(esters); poly(orthoesters); poly(esteramides); poly(anhydrides); poly(urethanes); poly(amides); cellulose ethers such as methyl cellulose, hydroxyethyl cellulose, hydroxypropyl methyl cellulose, and the like; cellulose esters such as cellulose acetate, cellulose acetate phthalate, cellulose acetate butyrate, and the like; poly(saccharides), proteins, gelatin, starch, gums, resins, and the like. These materials may be used alone, as physical mixtures (blends), or as copolymers.

Also polyacrylates, polymethacrylates, polybutylcyanoacrylates, polyalkylcyanoacrylates, polyarylamides, polyanhydrates, polyorthoesters, N,N-L-lysinediylterephthalate, polyanhydrates, desolvated biologically active agents or carbohydrates, polysaccharides, polyacrolein, polyglutaraldehydes and derivatives, copolymers and polymer blends.

In another embodiment of the present invention there is provided a method of encapsulating biologically active agents by producing particulate carriers comprising the steps of:
a) dissolving polybutylcyanoacrylate (PBCA) in an organic solvent to form a polymer solution;
b) adding an aqueous solution containing a biologically active agents to the polymer solution to form a primary emulsion of aqueous phase droplets in a continuous organic phase;
c) mixing the primary emulsion with an aqueous medium to form a secondary emulsion; and
d) allowing the organic phase to evaporate and thereby obtain particulate carriers comprising a hollow lumen containing biologically active agents in an aqueous phase.

In one embodiment the particulate carriers may be microspheres or nanoparticles. In a further embodiment the particulate carrier is a nanoparticle and the biologically active agent is a protein. In a further embodiment the particulate carrier is a nanoparticle and the biologically active agent is a peptide. In yet another embodiment the particulate carrier is a nanoparticle and the biologically active agent is an antigen binding molecule, for example a mAb or a dAb, or a combination thereof.

In a further embodiment of the method, step (d) additionally comprises the addition of gel forming polymers. In a further embodiment the gel forming polymer is agarose.

In one such embodiment the W/W ratio of protein to polymer may be 0.5% to 50% for example is at least about 0.5% or is at least about 1% or is at least about 2% or is at least about 5% or is at least about 7% or is at least about 10% or is at least about 11% or is at least about 14% or is at least about 20% or is at least about 40%, or is at least about 50%. For example when the protein is an antibody the antibody to polymer ratio may be at least about 14%, or when the protein is a domain antibody the domain antibody to polymer ratio may be at least about 11%.

In one embodiment of the present invention the W/W ratio of protein to total formulation may be 0.5% to 50% for example is at least about 5% or at least about 10% or at least about 15% or at least about 23% or at least about 50% or at least about 62% or at least about 71% or at least about 80%. For example when the protein is a peptide the peptide to total formulation ratio may be at least about 23% or when the protein is an antibody the antibody to total formulation ratio may be at least about 62% or when the protein is a domain antibody the domain antibody to total formulation ratio may be at least about 62%.

Examples of organic solvents suitable for use with the methods of the invention include but are not limited to water-immiscible esters such as ethyl acetate, isopropyl acetate, n-propyl acetate, isobutyl acetate, n-butyl acetate, isobutyl isobutyrate, 2-ethylhexyl acetate, ethylene glycol diacetate; water-immiscible ketones such as methyl ethyl ketone, methyl isobutyl ketone, methyl isoamyl ketone, methyl n-amyl ketone, diisobutyl ketone; water-immiscible aldehydes such as acetaldehyde, n-butyraldehyde, crotonaldehyde, 2-ethylhexaldehyde, isobutylaldehyde and propionaldehyde; water-immiscible ether esters such as ethyl 3-ethoxypropionate; water-immiscible aromatic hydrocarbons such as toluene xylene and benzene; water-immiscible halohydrocarbons such as 1,1,1 trichloroethane; water-immiscible glycol ether esters such as propylene glycol monomethyl ether acetate, ethylene glycol monoethyl ether acetate, ethylene glycol monobutyl ether acetate, diethylene glycol monobutyl ether acetate; water-immiscible phthalate plasticisers such as dibutyl phthalate, diethyl phthalate, dimethyl phthalate, dioctyl phthalate, dioctyl terephthalate, butyl octyl phthalate, butyl benzyl phthalate, alkyl benzyl phthalate; water-immiscible plasticisers such as dioctyl adipate, triethylene glycol di-2-ethylhexanoate, trioctyl trimellitate, glyceryl triacetate, glyceryl/tripropionin, 2,2,4-trimethyl-1,3-pentanediol diisobutyrate, methylene chloride, ethylacetate or dimethylsulfoxide, carbon tetrachloride, chloroform, cyclohexane, 1,2-dichloroethane, dichloromethane, diethyl ether, dimethyl formamide, heptane, hexane and other hydrocarbons, methyl-tert-butyl ether, pentane, toluene, 2,2,4-trimethylpentane, 1-octanol and its isomers or benzyl alcohol.

In one embodiment of the invention the solvent used in the methods of the invention will be selected from methylene chloride, ethylacetate or dimethylsulfoxide, carbon tetrachloride, chloroform, cyclohexane, 1,2-dichloroethane, dichloromethane, diethyl ether, dimethyl formamide, heptane, hexane and other hydrocarbons, methyl-tert-butyl ether, pentane, toluene, 2,2,4-trimethylpentane, 1-octanol and its isomers, benzyl alcohol.

The particulate carriers and methods in all aspects of the present invention as herein described may further comprise the addition of a surfactant such as but not limited to: sodium cholate, poloxamer 188 (pluronic F68), polyvinyl alcohol, polyvinyl pyrrolidone, polysorbate 80, dextrans. poloxamers, poloxamines, carboxylic acid esters of multifunctional alcohols, alkoxylated ethers, alkoxylated esters, alkoxylated mono-, di and triglycerides, alkoxylated phenols and diphenols, ethoxylated ethers, ethoxylated esters, ethoxylated triglycerides, substances of the GenapolR and BaukiR series, metal salts of fatty acids, metal salts of carboxylic acids, metal salts of alcohol sulfates, and metal salts of fatty alcohol sulfates and metal salts of sulfosuccinates and mixtures of two or more of said substances.

In a further embodiment the surfactant is sodium cholate, poloxamer 188 (pluronic F68), polyvinyl alcohol, polyvinyl pyrrolidone, polysorbate 80, dextrans.

The biologically active agent encapsulated in compositions of the present invention retains at least some biological activity on its release from the particulate carrier, for example, a proportion of the molecules in the composition may retain at least some ability to bind to their target and elicit a biological response on the release of the biologically active agent from the nanoparticles. Such binding can be measured in a suitable biological binding assay, examples of suitable assays. any assay suitable for measuring the binding in an in vitro assay of the biological molecule such as but not limited to ELISA or Biacore. In a further embodiment the composition retains at least 50% of its affinity for the target, or at least 70% or at least 90% of its affinity for the target when measured by a biological binding assay on release from the particles.

In one embodiment of the present invention there is provided a method of delivering a protein across a biological barrier such as the blood brain barrier by encapsulation of the protein in a nanoparticle.

In one embodiment of the present invention there is provided a method of delivering a protein encapsulated in a particulate carrier such as a microsphere by ocular delivery.

In another embodiment there is provided a pharmaceutical composition comprising a biologically active agent encapsulated in a particulate carrier of the present invention as herein described.

In a further embodiment there is provided a pharmaceutical composition comprising a protein encapsulated in the nanoparticles of the present invention as herein described.

In a further embodiment there is provided a pharmaceutical composition comprising a protein encapsulated in microspheres for ocular delivery as herein described.

In a further embodiment the compositions of the invention as herein described may be used to treat disorders or diseases of the Central nervous system, for example it may be used to treat Alzheimer's disease, Huntington's disease, bovine spongiform encephalopathy, West Nile virus encephalitis, Neuro-AIDS, brain injury, spinal cord injury, metastatic cancer of the brain, or multiple sclerosis, stroke.

In a further embodiment the composition may comprise anti-MAG antibodies for the treatment of stroke or other forms of neuronal injury.

In another embodiment the composition may comprise anti-NOGO antibodies for the treatment of stroke or other forms of neuronal injury or for example for the treatment or prophylaxis of neurodegenerative diseases such as Alzheimer's disease.

In a further embodiment the compositions of the invention as herein described may be used to treat disorders or diseases of the eye. In a further embodiment the compositions of the invention as herein described may be used to treat disorders such as but not limited to age related macular degeneration (neovascular/wet), diabetic retinopathy, retinal venous occlusive disease, uveitis, corneal neovascularisation or glaucoma.

In yet a further embodiment the composition is used to treat AMD (age related macular degeneration), for example wet AMD, or dry AMD.

In another embodiment of the present invention there is provided biologically active agents encapsulated in nanoparticles and or microspheres as described herein for use in medicine.

In one embodiment of the present invention there is provided the use of compositions of the invention as described herein in the manufacture of a medicament for the treatment of diseases of the central nervous system. In yet another embodiment there is provided the use of compositions of the invention as described herein in the manufacture of a medicament for the treatment of Alzheimer's disease. In yet a further embodiment there is provided the use of compositions of the invention as described herein in the manufacture of a medicament for the treatment of stroke or neuronal injury.

In another embodiment of the invention there is provided the use of compositions of the invention as described herein in the manufacture of a medicament for the treatment of ocular diseases such as for example in the manufacture of a medicament for the treatment of AMD.

The invention provides methods of treating diseases of the central nervous system using the compositions of the present invention. In a further embodiment there is provided a method of treating Alzheimer's disease using the compositions of the present invention. In yet another embodiment of the present invention there is provided a method of treating stroke or neuronal injury using the compositions of the present invention.

The invention also provides methods of treating ocular diseases using the compositions of the present invention. In a further embodiment there is provided a method of treating AMD using the compositions of the present invention.

### Definitions:

As used herein the term “particle forming substance” is used to describe any monomer capable of polymerising to form an insoluble particle. The particle forming substance will be soluble in a solvent when not polymerised.

The term “particulate carrier” as used throughout this specification is used to cover both nanoparticles and microspheres. “Microspheres” are particles composed of various natural and synthetic materials with diameters larger than 1 μm whereas “nanoparticles” as used herein are submicron sized particles such as for example 1-1000 nm.

The terms particulate carrier, nanoparticles and microspheres as used herein denotes a carrier structure which is biocompatible and sufficiently resistant to chemical and/or physical destruction by the environment of use such that a sufficient amount of the particles remain substantially intact after entry in to the human or animal body following administration and for sufficient time so as to be able to reach the desired target organ or tissue e.g. the brain or the eye.

The term “Biologically active agent” as used herein is a term used to indicate that the molecule must be capable of at least some biological activity when reaching their desired target.

The term “protein” as used throughout this specification for encapsulation in particulate carriers includes proteins having a molecular weight of at least 11 kDa, or at least 12 kDa, or at least 50 kDa, or at least 100 kDa, or at least 150 kDa or at least 200 kDa. Proteins for encapsulation may also be of considerable length such as at least 70 amino acids in length or at least 100 amino acids in length or at least 150 amino acids in length or at least 200 amino acids in length.

The term “peptide” as used throughout this specification for encapsulation in particulate carriers includes shorter sequences of amino acids having a molecular weight of no more than about 10 kDa, or no more than about 8 kDa, or no more than about 5 kDa, or no more than about 2 kDa or no more than about 1 kDa or is less than 1 Kda. Peptides for encapsulation are no more than 70 amino acids in length or are no more than 50 amino acids in length, or are no more than are no more than 40 amino acids in length, or are no more than 20 amino acids in length or are less than 10 amino acids in length.

The phrase “immunoglobulin single variable domain” refers to an antibody variable domain (V_{H}, V_{HH}, V_{L}) that specifically binds an antigen or epitope independently of a different V region or domain. An immunoglobulin single variable domain can be present in a format (e.g., homo- or hetero-multimer) with other, different variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is the same as an “immunoglobulin single variable domain” which is capable of binding to an antigen as the term is used herein. An immunoglobulin single variable domain may be a human antibody variable domain, but also includes single antibody variable domains from other species such as rodent (for example, as disclosed in WO 00/29004, nurse shark and Camelid V_{HH} dAbs. Camelid V_{HH} are immunoglobulin single variable domain polypeptides that are derived from species including camel, llama, alpaca, dromedary, and guanaco, which produce heavy chain antibodies naturally devoid of light chains. Such V_{HH} domains may be humanised according to standard techniques available in the art, and such domains are still considered to be “domain antibodies” according to the invention. As used herein “V_{H} includes camelid V_{HH} domains.

The term “Light scattering techniques” as used herein is a means used to determine the size distribution profile of small particles in solution—such as for example dynamic light scattering which is used to measure nanoparticles and static light scattering used to measure microspheres.

The term “Dynamic light scattering” (DLS) as used herein is a method which utilises the light scattered by particle dispersions to derive information on the size of the particles. Dynamic light scattering relies on the fact that when in liquid suspension, the Brownian motion of particles is dependent on particle size and that the Brownian motion of the particles produces fluctuations in the intensity of light scattered from a particle sample. The particle diameter is derived by analysing these fluctuations by means of a correlation function. The Stokes-Einstein equation is then applied to yield the mean hydrodynamic diameter of the particles.

A multi-exponential analysis can produce a size distribution, providing insight into the presence of different species inside a sample. DLS is generally accepted for the analysis of nanoparticles (approximately 1000 nm in diameter).

“Low angle laser light scattering” as used throughout the specification is sometimes referred to as Laser diffraction. Laser diffraction relies on the fact that the diffraction angle is inversely proportional to particle size. The method utilises the full Mie theory which completely solves the equations for the interaction of light with matter. Laser diffraction can be used for the analysis of nanoparticles and microparticles (0.02 to 2000 micrometers in diameter).

The term “Blood brain barrier” (BBB) as used herein is a membranic structure that acts primarily to protect the brain from chemicals in the blood, while still allowing essential metabolic function. It is composed of cerebral microvascular endothelial cells, which are packed very tightly in brain capillaries. This higher density restricts passage of substances from the bloodstream much more than endothelial cells in capillaries elsewhere in the body.

Throughout this specification the percentage drug loading is defined as the percentage of weight of drug per weight of material used in the particle formulation (polymer weight) W/W.

% drug loading=(weight of drug/weight of material used in the particle formulation)×100%.

### EXAMPLES

### Example 1

### Polymerisation of BCA (Butylcyanoacrylate) Monomer

A rapid polymerisation reaction in organic solvent was used to form the polymer: BCA monomer (200 μl, Vetbond, 3M) was added to 1 ml absolute ethanol in a 25 ml beaker with slow swirling. The resulting solution was gently mixed until the polymerisation reaction was initiated. The polymerisation reaction resulted in the formation of a white solid dispersion. The mixing of the dispersion was stopped as soon as the reaction mixture became too viscous to agitate.

The ethanol in the reaction mixture was then allowed to evaporate in the fume-hood for at least 1 hour. Following evaporation of the ethanol, a cracked white solid cake was obtained. The solid was collected and used in the nanoparticle preparation process.

### Example 2

### Preparation of Hollow Nanoparticles by the Double Emulsion Method

The PBCA polymer was dissolved in dichloromethane at a concentration of 1% w/v and used to prepare hollow PBCA nanoparticles by emulsification into a double emulsion (water in oil in water, w/o/w) as follows:

(i) Primary emulsion (w/o)

Inner phase (w): 5% sodium cholate (SIGMA) in water or buffer, prepared by mixing:

500 μl water or buffer, and

500 μl sodium cholate (10% w/v stock solution).

The total volume of the inner aqueous phase was 1 ml. The solution was kept on ice until it was time to use it. Each solution was drawn into a 1 ml insulin syringe (Terumo 1 ml, BD microlance needle 19 G 1.5″) prior to use.

Outer (organic) phase (o): PBCA polymer (1% w/v) in dichloromethane (“DCM: Fischer).

The organic phase (PBCA polymer in DCM, 6 ml) was poured into a 10 ml beaker (resting on ice to keep cool) and the probe of the homogeniser was inserted (Ultra-Turrax, T25, 50 ml probe). The solution was covered with parafilm (attached to the beaker and probe) and homogenised at a speed of 25,000 rpm.

### Formation of a Primary Emulsion:

As soon as the homogeniser reached the top speed, the inner aqueous phase was added by injecting inside the solution close to the probe. The resulting emulsion was homogenised for 2 minutes (on ice) and then transferred to a glass syringe (SGE, 25 ml, gas-tight, suitable for organic solvents, P/N 009462 25MDR-LL-GT, Batch # F06-A2190, fitted with blunt 5 cm 2R2 needle, 0.7 mm ID).

Secondary emulsion (w/o/w)

Inner phase (w/o): primary emulsion from homogenisation step described above.

Outer phase (w): sodium cholate (1.25% w/v) in water.

### Formation of the Secondary Emulsion:

The primary, single emulsion (w/o) was used to form a double emulsion (w/o/w) by addition to a secondary aqueous phase (1.25% w/v sodium cholate) with homogenisation. The sodium cholate solution (1.25% w/v, 30 ml) was transferred to a tall 50 ml beaker (resting on ice to keep emulsion cool) and the probe of a Silverson L4RT homogeniser was inserted (¾ inch probe, high emulsor screen). The solution was covered with parafilm (attached to the beaker and probe) and homogenised at a speed of 8,000 rpm. The primary emulsion was injected into the solution close to the probe as soon as the 8,000 rpm speed was reached. The resulting emulsion was homogenised for 6 minutes.

The double emulsion that was formed was transferred to a short 50 ml beaker and the organic phase allowed to evaporate in the fume hood under constant stirring (IKA magnetic stirrer, setting 4) for 3 hours.

### Washing of the Nanoparticles by Centrifugation:

Following removal of the organic solvent, the nanoparticles that were formed were washed once by centrifugation at 16,200 rpm and re-suspended in water (10 ml).

### Example 3

### Confirmation of Nanoparticle Formation by Dynamic Light Scattering

The formation of nanoparticles was confirmed by sizing using dynamic light scattering (DLS). The particles were analysed using a Brookhaven Instruments corporation particle size analyser (BIC 90 plus). Sizing by DLS showed that nanoparticles of a mean hydrodynamic diameter of around 260 nm had formed.

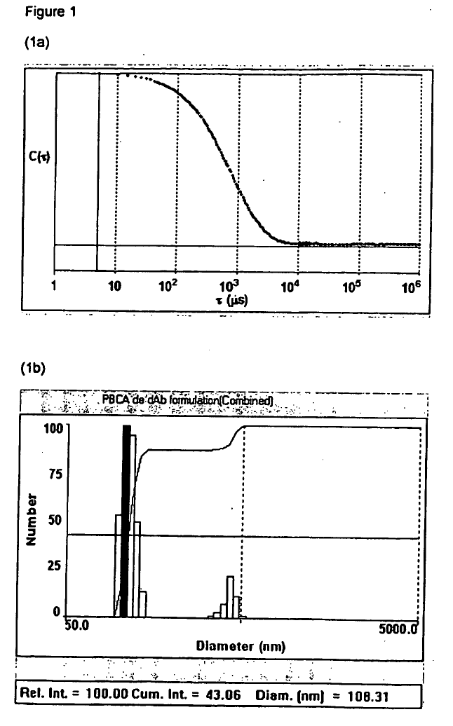

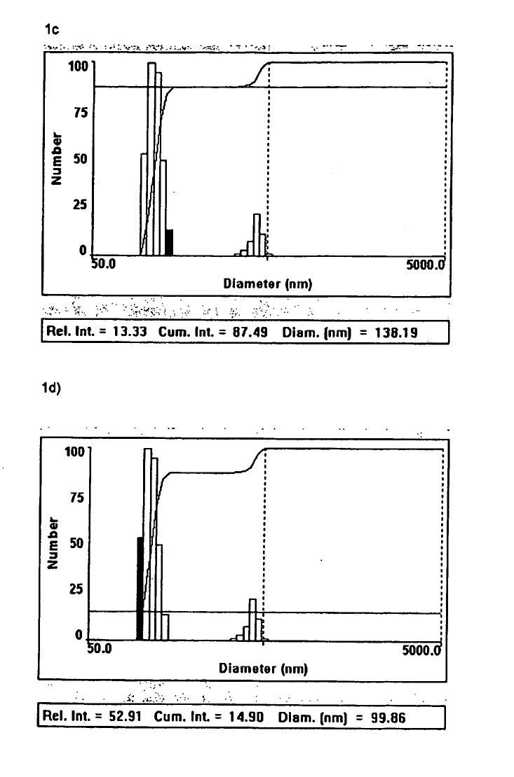

FIG. 1 shows the sizing data obtained by DLS that indicate the presence of nanoparticles in suspension. The derived data suggests that the majority of the particles are small (FIGS. 1b-d). The results suggest that approximately 87% of the particle population has a diameter below 138.19 nm. This suggests that around 72% of the particle population lies within a diameter range from 100 to 138 nm.

FIG. 1.—Sizing data obtained by DLS that indicate the presence of nanoparticles in suspension.

FIG. 1(a) Correlogram obtained following analysis of a nanoparticle suspension by dynamic light scattering. According to the data obtained, the mean hydrodynamic diameter of the particles was 262.6 nm and the polydispersity index 0.262.

FIG. 1(b) Multimodal size distribution (derived data) of the nanoparticles plotted to depict the distribution of the particle population (number) over a range of sizes. The majority of the particle population appears to have a diameter of around 108 nm.

FIG. 1(c) Multimodal size distribution (derived data) of the nanoparticles plotted to depict the distribution of the particle population (number) over a range of sizes. The data suggests that 87.5% of the particle sample possesses a diameter of around 138.19 nm or lower.

FIG. 1(d) Multimodal size distribution (derived data) of the nanoparticles plotted to depict the distribution of the particle population (number) over a range of sizes. The data suggests that 14.9% of the particle sample possesses a diameter of around 99.86 nm or lower.

### Example 4

### Analysis of Nanoparticles—Confirmation of Nanoparticle Formation and Hollow Morphology by Electron Microscopy

In order to confirm that the particles were formed and that they were hollow, samples were visualised by electron microscopy. Nanoparticle suspensions were examined by transmission electron microscopy (TEM). Freeze-dried nanoparticles were analysed by scanning electron microscopy (SEM). Analysis by both microscopy techniques confirmed the formation of nanoparticles. SEM showed that stable nanoparticles were formed. TEM confirmed that the nanoparticles were hollow, possessing an aqueous core surrounded by a PBCA polymer wall.

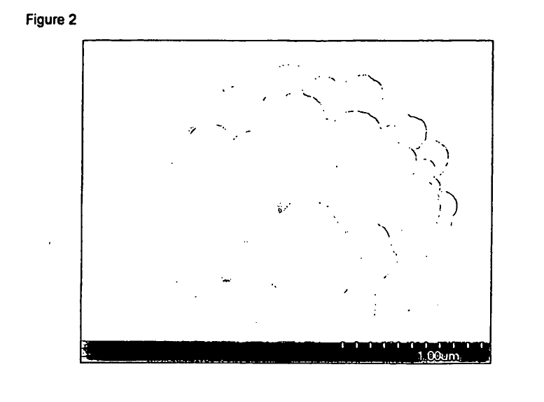

FIG. 2 shows nanoparticles analysed by SEM

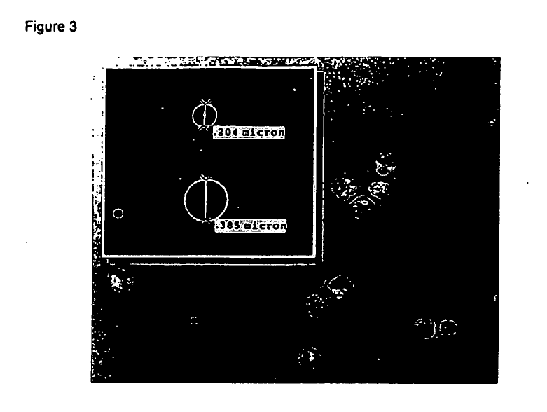

FIG. 3 shows an image of hollow nanoparticles by TEM, with a superimposed image of solid PBCA nanoparticles for comparison.

### Example 5

### Encapsulation of Monoclonal Antibody (Human Anti-CD23) within the Hollow PBCA Nanoparticles

Monoclonal antibody (human anti-CD 23 mAb) was entrapped within the aqueous core of the nanoparticles by inclusion into the inner aqueous phase of the homogenisation process. A solution of the antibody was used to prepare the primary emulsion (w/o), which was then homogenised with the secondary aqueous phase to form the double emulsion (w/o/w) as follows:

(iii) Primary emulsion (w/o)

Inner phase (w): anti-CD23 mAb (600 μg in 5% sodium cholate (SIGMA), prepared by mixing:
78 μl mAb solution (7.2 mg/ml)
344 μl H2O
500 μl sodium cholate solution (10% w/v stock solution)

The total volume of the inner aqueous phase was 1 ml. The solution was kept on ice until it was time to use it. Each solution was drawn into a 1 ml insulin syringe (Terumo 1 ml, BD microlance needle 19 G 1.5″) prior to use.

Outer phase (o): PBCA polymer (1% w/v) in dichloromethane (Fischer).

The organic phase (PBCA polymer in DCM, 6 ml) was poured into a 10 ml beaker (resting on ice to keep cool) and the probe of the homogeniser was inserted (Ultra-Turrax, T25, 50 ml probe). The solution was covered with parafilm (attached to the beaker and probe) and homogenised at a speed of 25,000 rpm.

### Formation of a Primary Emulsion:

As soon as the homogeniser reached the top speed, the inner aqueous phase was added by injecting inside the solution close to the probe. The resulting emulsion was homogenised for 2 minutes (on ice) and then transferred to a glass syringe (SGE, 25 ml, gas-tight, suitable for organic solvents, P/N 009462 25MDR-LL-GT, Batch # F06-A2190, fitted with blunt 5 cm 2R2 needle, 0.7 mm ID).

(iv) Secondary emulsion (w/o/w)

Inner phase (w/o): primary emulsion from homogenisation step described above.

Outer phase (w): sodium cholate (1.25% w/v) in water.

### Formation of the Secondary Emulsion:

The primary, single emulsion (w/o) was used to form a double emulsion (w/o/w) by addition to a secondary aqueous phase (1.25% w/v sodium cholate) with homogenisation. The sodium cholate solution (1.25% w/v, 30 ml) was transferred to a tall 50 ml beaker (resting on ice to keep emulsion cool) and the probe of a Silverson L4RT homogeniser was inserted (¾ inch probe, high emulsor screen). The solution was covered with parafilm (attached to the beaker and probe) and homogenised at a speed of 8,000 rpm. The primary emulsion was injected into the solution close to the probe as soon as the 8,000 rpm speed was reached. The resulting emulsion was homogenised for 6 minutes.

The double emulsion that was formed was transferred to a short 50 ml beaker and the organic phase allowed to evaporate in the fume hood under constant stirring (IKA magnetic stirrer, setting 4) for 3 hours.

### Washing of the Nanoparticles by Centrifugation:

The resulting nanoparticles were pelleted by centrifugation to separate free from encapsulated antibody. Both pellet (entrapped antibody) and supernatant (free antibody) were analysed by total protein assay in order to determine the encapsulation efficiency.

The encapsulation efficiency was found to be 52%, when a total amount of 600 μg antibody was used. The efficiency of encapsulation was found to be sufficiently high to permit the delivery of potentially therapeutic amounts of antibody without exceeding the maximum tolerated dose of PBCA polymer (50 mg/kg in the mouse). Moreover, it was later possible to prepare particles containing a different monoclonal antibody, human anti-IL13, which suggests that the method is applicable to any water-soluble biopharmaceutical.

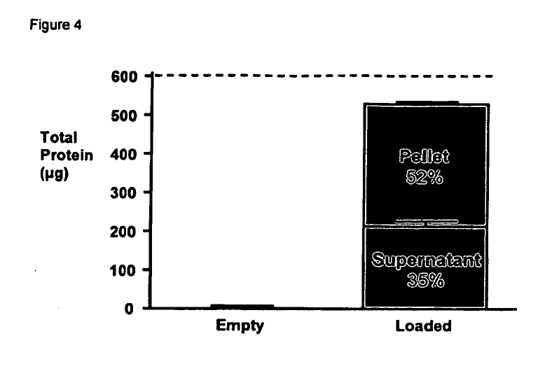

FIG. 4 show the results obtained from the encapsulation efficiency measurements.

### Example 6

### Release of Monoclonal Antibody from the Nanoparticles

In addition to achieving efficient encapsulation of a biopharmaceutical, it was necessary to demonstrate that the material could be released from the particles following administration and that it retained its activity. The release of active antibody from the particles was initially investigated in vitro by degradation of the particles followed by detection of any released antibody by ELISA. In order to release the encapsulated antibody, particles were treated with a butyl esterase (from porcine liver, SIGMA), which has been reported to cleave the butyl ester of the PBCA polymer. During the reaction (Ringers solution, pH 7.0, 37° C.), samples were taken at different time points (0, 1, 2, 3, 4 and 24 h) and analysed for the presence of active antibody by ELISA.

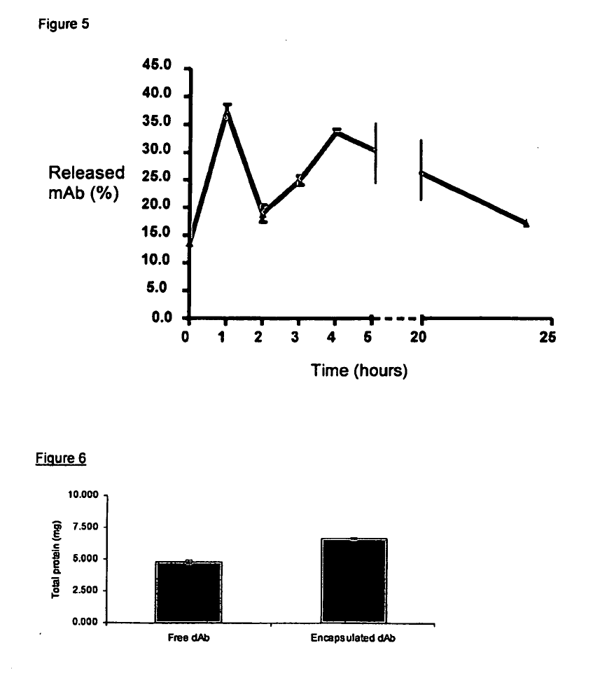

FIG. 5 shows the release profile obtained following enzymatic degradation of the particles and analysis of the released enzyme by ELISA.

### Example 7

### Encapsulation of Domain Antibody (Anti-Hen Egg Lysozyme dAb) within the Hollow PBCA Nanoparticles

Domain antibody (anti-hen egg lysozyme dAb) was entrapped within the aqueous core of the nanoparticles by inclusion into the inner aqueous phase of the homogenisation process. In this case, the inner aqueous phase was prepared by mixing 0.5 ml of a 20 mg/ml solution of dAb (10 mg protein) and 0.5 ml of a stabiliser solution (sodium cholate, 10% w/v). The nanoparticles were then prepared by the double emulsion process as described in example 4. The resulting nanoparticles were pelleted by centrifugation to separate free from encapsulated antibody. Both pellet (entrapped antibody) and supernatant (free antibody) were analysed by total protein assay (bicinchoninic acid assay) in order to determine the encapsulation efficiency. The results of the analysis are shown in FIG. 6. The amount of encapsulated dAb was found to be 6.66 mg. The amount of free dAb was 4.83 mg. The efficiency of encapsulation was therefore around 66.6%, with the loading efficiency at 11.1%. It was therefore possible to efficiently encapsulate milligram amounts of protein in the hollow PBCA nanoparticles using the double emulsion method.

### Example 8

### Preparation of PBCA Nanoparticles by the HIP Process

The nanoparticles were prepared by adding 100 μl BCA monomer to an organic phase containing solubilised HIP ion (docusate sodium, 3.058-6.116% w/v in 1 ml dichloromethane). The resulting solution was pipetted into an aqueous phase (1% w/v dextran, 0.2% w/v pluronic F68, 10 ml, pH 7.0) with homogenisation at 7,000 using a Silverson L4RT homogeniser. Exposure to the neutral pH of the aqueous phase resulted in rapid polymerisation of the BCA monomer to form PBCA polymer. The emulsion that was formed was homogenised for 30 seconds and then incubated in a fume hood for 3 hours to allow the organic solvent to evaporate and nanoparticles to form. The resulting nanoparticle suspension was stored at 4° C.

### Example 9

### Confirmation of Nanoparticle Formation by Dynamic Light Scattering

The formation of PBCA nanoparticles by the HIP process was confirmed by sizing using dynamic light scattering (DLS). The particles were analysed using a Brookhaven Instruments corporation particle size analyser (BIC 90 plus). Sizing by DLS showed that nanoparticles of a mean hydrodynamic diameter of around 290 nm had formed.

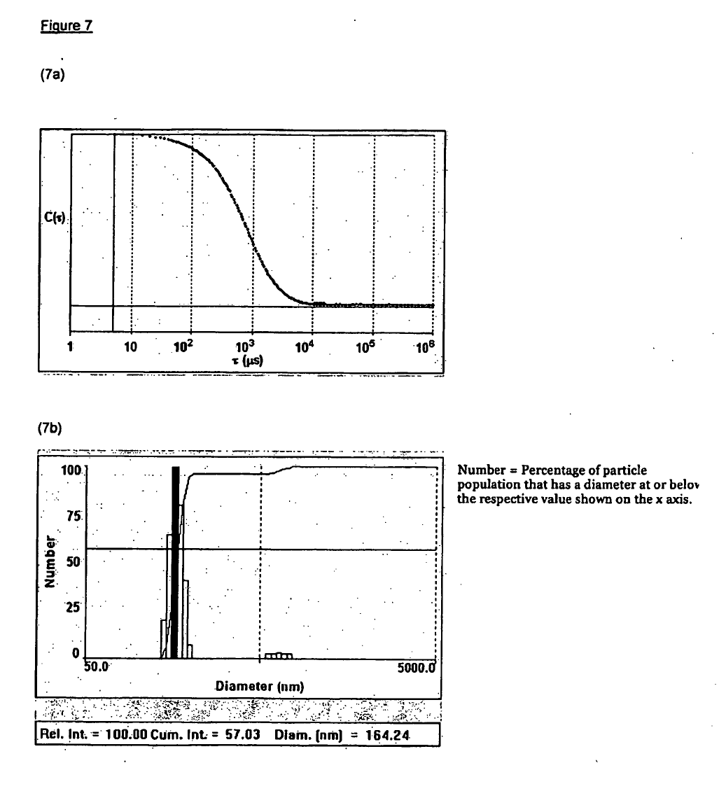

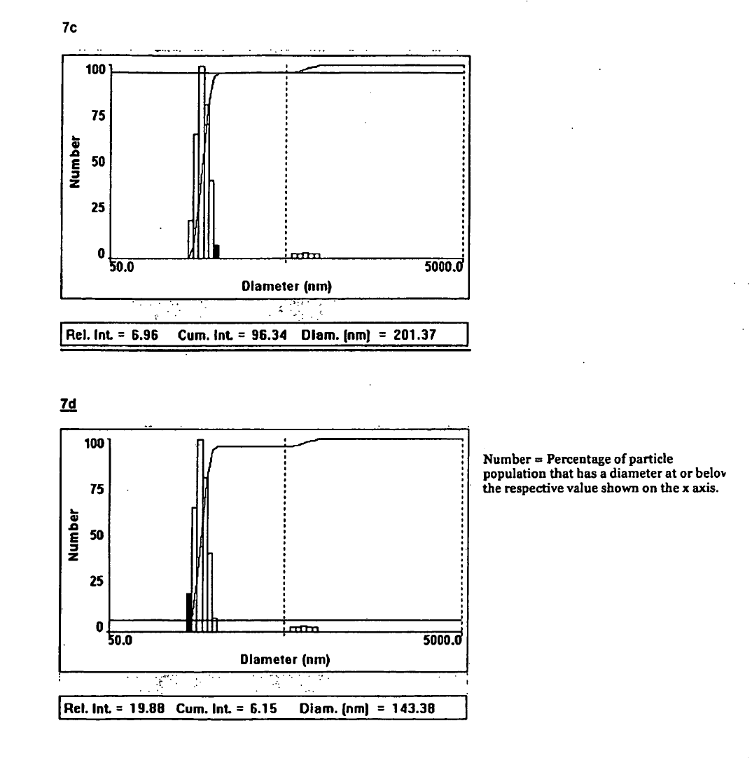

FIG. 7 shows the sizing data obtained by DLS that indicate the presence of nanoparticles in suspension. The derived data suggest that the majority of the particles are small (FIG. 7b-d). The results suggest that approximately 96% of the particle population has a diameter below 201.37 nm. Approximately 6% of the particle population has a diameter below 143.38 nm. This suggests that around 90% of the particle population lies within a diameter range from 143 to 201 nm.

FIG. 7(a) Correlogram (raw data) obtained following analysis of a nanoparticle suspension by dynamic light scattering. According to the data obtained, the mean hydrodynamic diameter of the particles was 291.4 nm and the polydispersity index 0.242.

FIG. 7(b) Multimodal size distribution (derived data) of the nanoparticles plotted to depict the distribution of the particle population (number) over a range of sizes. The majority of the particle population appears to have a diameter of around 164 nm.

FIG. 7(c) Multimodal size distribution (derived data) of the nanoparticles plotted to depict the distribution of the particle population (number) over a range of sizes. The data suggests that 96.4% of the particle sample possesses a diameter of around 201.37 nm or lower.

FIG. 7(d) Multimodal size distribution (derived data) of the nanoparticles plotted to depict the distribution of the particle population (number) over a range of sizes. The data suggests that 6.2% of the particle sample possesses a diameter of around 143.38 nm or lower.

### Example 10

### Solubilisation of Peptides into the Organic Phase using the HIP Process and Encapsulation into PBCA Nanoparticles

A solution of the hexapeptide dalargin was prepared by dissolving 30-60 mg of the peptide into 3 ml of CaCl2 (18.3 mM) and lowering the pH to 3.05 by addition of concentrated HCl (2M). The resulting solution (500 μl, 10-20 mg/ml, and total amount of peptide 5-10 mg) was added to a solution of the HIP agent docusate sodium in dichloromethane (1 ml, 3.058-6.116% w/v). The volume of HIP solution used was twice that of the peptide solution. The molar ratio of HIP:peptide was 10:1 with 5 mg peptide and 5:1 with 10 mg peptide. The organic and aqueous phases were mixed by vortexing at maximum speed for 1 minute. The resulting suspension was then centrifuged to separate the two phases. The organic layer (containing solubilised peptide) was collected and used to prepare nanoparticles.

In order to confirm that the process had been successful at solubilising the peptide into the organic phase, the amount of peptide remaining in the aqueous phase was determined. Analysis by LC-MS and Edman sequencing showed that at least 99% of the peptide had been successfully extracted into the organic phase.

### Example 11

### Encapsulation of Peptide within the PBCA Nanoparticles

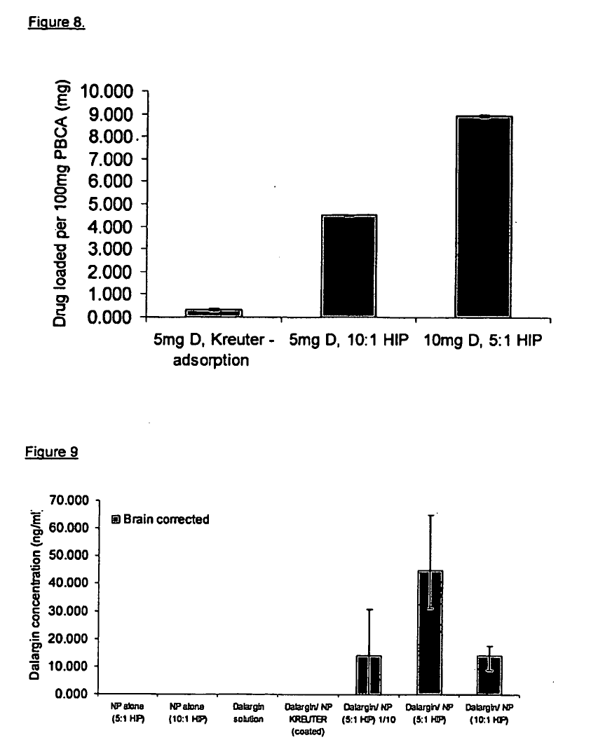

The nanoparticles were prepared by adding 100 μl BCA monomer to the organic phase containing solubilised peptide and HIP (1 ml). The resulting solution was pipetted into an aqueous phase (1% w/v dextran, 0.2% w/v pluronic F68, 10 ml, pH 7.0) with homogenisation at 7,000 using a Silverson L4RT homogeniser. Exposure to the neutral pH of the aqueous phase resulted in rapid polymerisation of the BCA monomer to form PBCA polymer. The emulsion that was formed was homogenised for 30 seconds and then incubated in a fume hood for 3 hours to allow the organic solvent to evaporate and nanoparticles to form. The resulting nanoparticles were centrifuged to remove any free peptide and re-suspended in water or PBS. The encapsulation efficiency was determined by analysing the particles by LC-MS. It was found that approximately 90% of the peptide dose was encapsulated, even when high peptide amounts were used (10 mg). Comparison of the amounts of encapsulated peptide achieved with the HIP to those achieved by the common method of adsorption onto the particle surface clearly demonstrated the superiority of the HIP-PBCA process (FIG. 8). When the adsorption method was used, a mere 1.5% of the peptide dose was loaded onto the particles.

### Example 12

### Evaluation of the HIP-PBCA Nanoparticle Delivery System In Vivo (Mouse Model)

The ability of the HIP-PBCA nanoparticles to deliver their peptide load to the brain was determined in vivo in the mouse model. HIP-PBCA nanoparticles containing encapsulated dalargin using the HIP process were compared to HIP-PBCA nanoparticles that had had the peptide adsorbed onto the particle surface as reported by Kreuter et al. The nanoparticles were prepared for brain delivery via the intravenous route by coating their surface with polysorbate 80 surfactant. Briefly, the nanoparticles were incubated in PBS containing 1% w/v surfactant for 30 minutes prior to injection. The surfactant has been reported in the literature to indirectly target the nanoparticles to the brain by promoting adsorption of serum apolipoproteins onto the nanoparticle surface. This allows the particles to bind to the apolipoprotein receptor on the blood brain barrier and transcytose to reach the brain. The following formulations were compared:
1. HIP-PBCA nanoparticles alone (5:1 HIP content)
2. HIP-PBCA nanoparticles alone (10:1 HIP content)
3. Dalargin in solution (2.0 mg/kg)
4. HIP-PBCA nanoparticles with dalargin adsorbed onto the surface (2.0 mg/kg total dose used in formulation)
5. HIP-PBCA nanoparticles (5:1 HIP:dalargin molar ratio) with dalargin encapsulated (2.0 mg/kg total dose used in formulation)
6. HIP-PBCA nanoparticles (5:1 HIP:dalargin molar ratio) with dalargin encapsulated (2.0 mg/kg total dose used in formulation)—the same formulation as above, but injected at 1/10 of the dose.
7. HIP-PBCA nanoparticles (10:1 HIP:dalargin molar ratio) with dalargin encapsulated (2.0 mg/kg total dose used in formulation)

The mice were sacrificed at 20 minutes following injection, and the brains and blood samples collected and analysed for the presence of peptide by LC-MS-MS. The brain data were corrected for blood contamination assuming a blood contamination of 15 μl per gram of brain. The results obtained are shown in FIG. 9:

The results of the in vivo study suggest that encapsulation of the peptide within the core of HIP-PBCA nanoparticles using the HIP process, is superior to adsorption of the peptide on the particle surface.

### Example 13

### Effect of pH on the Encapsulation Efficiency of Dalargin in HIP-PBCA Nanoparticles

In the prior art, the PBCA nanoparticles are formed by slow polymerisation of the BCA monomer in an acidic water in oil emulsion, where the pH of the aqueous phase is around 2.0 (0.01 N HCl). The polymerisation reaction under acidic conditions requires a period of at least 3 hours to reach completion. This invention employs a neutral pH to allow rapid polymerisation. The aqueous phase that is used is phosphate buffered saline (PBS, pH 7.2). At neutral pH, the BCA monomer is known to polymerise rapidly (within seconds). As a result, the production of HIP-PBCA nanoparticles requires the very quick formation of an emulsion. This is achieved in this invention by means of homogenisation at high speed (7,500 rpm or higher) using a Silverson L4RT homogeniser. It was hypothesized that a faster polymerisation reaction at neutral pH would improve the encapsulation efficiency, by quickly entrapping the peptide in the particles. In contrast, prolonged polymerisation could lead to gradual loss of peptide from the emulsion into the aqueous phase. To test the hypothesis, nanoparticles were prepared, using the HIP-PBCA process, by emulsifying the BCA monomer with extracted peptide in either PBCS or the original medium of the prior art, 0.01 N HCl. Both the acidic and neutral aqueous phases contained the required stabilisers (0.2% pluronic F68, 1% dextran). The nanoparticles were prepared following the procedure described in example 3. The amount of peptide used per formulation was 5 mg. The following formulations were prepared (one preparation each):
1. HIP-PBCA nanoparticles alone (35:1 HIP content), pH2
2. HIP-PBCA nanoparticles alone (35:1 HIP content), pH7
3. HIP-PBCA nanoparticles (35:1 HIP:dalargin molar ratio) with dalargin encapsulated (5.0 mg input), pH2
4. HIP-PBCA nanoparticles (35:1 HIP:dalargin molar ratio) with dalargin encapsulated (5.0 mg input), pH7
5. HIP-PBCA nanoparticles (10:1 HIP:dalargin molar ratio) with dalargin encapsulated (5.0 mg input), pH2
6. HIP-PBCA nanoparticles (10:1 HIP:dalargin molar ratio) with dalargin encapsulated (5.0 mg input), pH7
7. HIP-PBCA nanoparticles (5:1 HIP:dalargin molar ratio) with dalargin encapsulated (5.0 mg input), pH2
8. HIP-PBCA nanoparticles (5:1 HIP:dalargin molar ratio) with dalargin encapsulated (5.0 mg input), pH7

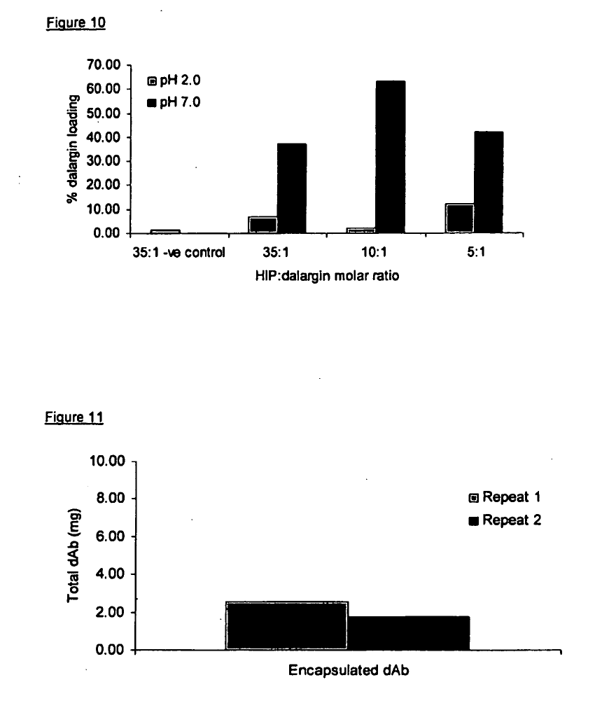

The nanoparticle formulations were centrifuged to remove any free peptide and re-suspended in water or PBS. The encapsulation efficiency was determined by breaking up the particles in 10 mM NaOH (overnight incubation at room temperature) and then analysing by LC-MS. The results obtained are shown in FIG. 10.

The results support the hypothesis that rapid formation of the PBCA polymer at neutral pH would result in higher peptide encapsulation efficiency than slow formation of the polymer at acidic pH as is the case in the prior art. Despite loss of some of the peptide due to degradation by the treatment with NaOH, the results obtained clearly show the benefits of forming the particles at neutral pH. At a HIP:dalargin ratio of 10:1, 63.23% of the input peptide was entrapped into the nanoparticles when the particles were formed at pH 7. At pH 2, the encapsulation efficiency was significantly lower at 2.36%. Overall, the encapsulation efficiency was higher when the particles when the particles were prepared at pH 7 than when they were prepared at pH 2.

### Example 14

### Encapsulation of Domain Antibody in PBCA Nanoparticles using the HIP Process

A domain antibody (anti-hen egg lysozyme dAb) was formulated in PBCA nanoparticles following the procedure described in example 3. The amount of protein used in the formulation was 10 mg. A total of two formulations were prepared. In order to determine the amount of encapsulated dAb, the particles were centrifuged to remove any free protein and then analysed by Edman sequencing. In addition to sequence information, Edman sequencing can also be used to provide quantitative information. The process involves harsh chemical treatment which destroys the particle and allows detection of the encapsulated material. The results obtained are shown in FIG. 11. The results suggest that it is possible to encapsulate a larger molecule using the HIP-PBCA process, but at a lower efficiency. However, it may be possible to increase the efficiency of encapsulation by optimising the protocol for use with the domain antibody. With the current protocol, which has been optimised for dalargin, it was possible to encapsulate approximately 2.56 mg of the 10 mg used. This amounts to an encapsulation efficiency of 25.6%, which is high for a single emulsion process where a protein is entrapped within a hydrophobic particles matrix.

### Example 15

### Encapsulation of Monoclonal Antibody (Anti-IL-13 mAb) within the Hollow PBCA Nanoparticles

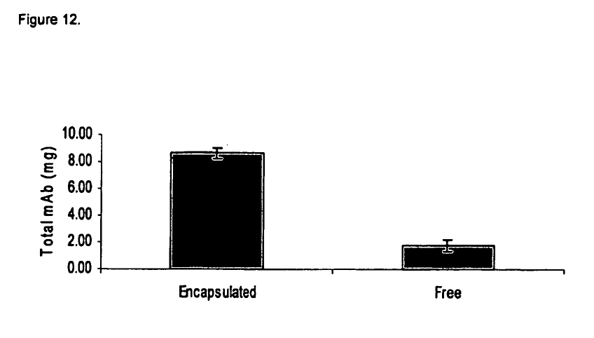

Monoclonal antibody (anti-IL-13 mAb) was entrapped within the aqueous core of the nanoparticles by inclusion into the inner aqueous phase of the homogenisation process. The inner aqueous phase was prepared by mixing 0.5 ml of a 20 mg/ml solution of mAb (10 mg protein) and 0.5 ml of a stabiliser solution (sodium cholate, 10% w/v). The nanoparticles were then prepared by the double emulsion process as described in Example 5. The resulting nanoparticles were pelleted by centrifugation to separate free from encapsulated antibody. Both pellet (entrapped antibody) and supernatant (free antibody) were analysed by total protein assay (bicinchoninic acid assay) in order to determine the encapsulation efficiency. The results of the analysis are shown in FIG. 12. The amount of encapsulated mAb was found to be 8.62 mg. The amount of free mAb was 1.79 mg. The efficiency of encapsulation was 86.2%, with the loading efficiency 14.4% w/w.

1. Nanoparticles comprising a particle forming substance and a protein for delivery of the protein from the blood to the brain across the blood brain barrier.

2. Nanoparticles as claimed in claim 1 wherein the protein is an antigen binding molecule.

3. Nanoparticles as claimed in claim 2 wherein the protein comprises an antibody.

4. Nanoparticles as claimed in claim 2 wherein the protein comprises a dAb.

5. Nanoparticles according to any preceding claim wherein the protein binds to a target found in the central nervous system.

6. Nanoparticles as claimed in claim 5 wherein the protein binds to NOGO, β-amyloid or Myelin associated glycoprotein.

7. A composition comprising the nanoparticles of any preceding claim wherein at least 90% of the nanoparticles by number in the composition are within the range of about 1 nm to about 1000 nm in diameter as measured by dynamic light scattering techniques.

8. A composition comprising the nanoparticles as claimed in any one of claims 1 to 6 wherein at least 90% of the nanoparticles by number in the composition are within the range of about 1 nm to about 400 nm in diameter as measured by dynamic light scattering techniques.

9. A composition comprising the nanoparticles as claimed in any one of claims 1 to 6 wherein at least 90% of the nanoparticles by number in the composition are within the range of about 1 nm to about 150 nm in diameter as measured by dynamic light scattering techniques.

10. A composition as claimed in any one of claims 7 to 9 wherein the average particle size is about 40 nm to about 100 nm.

11. A method of encapsulating biologically active agents in a particulate carrier comprising the steps of:
a) solubilising a biologically active agent in the presence of a hydrophobic ion pairing (HIP) agent and in an organic solvent;
b) dissolving a monomer of a polymer forming substance in the organic phase formed in (a);
c) forming an emulsion of the organic phase formed in (b) in a continuous aqueous phase to allow polymerisation of the monomer; and
d) obtaining a particulate carrier formed from the emulsion.

12. The method of claim 11 wherein the particulate carrier is a nanoparticle.

13. The method of claim 11 or 12 wherein the biologically active agent is a protein.

14. The method of claim 11 wherein the particulate carrier is a nanoparticle and the biologically active agent is a protein.

15. The method of claim 13 or 14 wherein the protein is insoluble in the organic phase without the presence of hydrophobic ion pairing agents.

16. The method of any one of claims 11 to 15 wherein the continuous aqueous phase in step (c) has a pH of about 6 or higher.

17. The method of any one of claims 11 to 16, wherein the HIP agent is an anionic HIP agent when the protein is cationic.

18. The method of claim 17 wherein the HIP agent is ducosate sodium.

19. The method of any one of claims 11 to 16, wherein the HIP agent is a cationic HIP agent when the protein is anionic.

20. The method of claim 19 wherein the HIP agent is dimethyldioctadecyl-ammonium bromide (DDAB18); 1,2-dioleoyloxy-3(trimethylammonium)propane (DOTAP); or cetrimonium bromide (CTAB).

21. A method of making a nanoparticle as claimed in claim 1 comprising the steps described in any one of claims 11 to 20.

22. The method of any one of claims 11 to 21 wherein the protein to polymer ratio in the nanoparticle is at least 1.5% W/W.

23. The method of any one of claims 11 to 22 wherein the monomer comprises alkylcyanoacrylate (ACA).

24. The method of claims 23 wherein the monomer comprises butylcyanoacrylate (BCA)

25. A method of encapsulating biologically active agents in particulate carriers for ocular delivery comprising the steps of:
a) dissolving a polymer in an organic solvent to form a polymer solution;
b) adding an aqueous solution containing a biologically active agents to the polymer solution to form a primary emulsion of aqueous phase droplets in a continuous organic phase;
c) mixing the primary emulsion with an aqueous medium to form a secondary emulsion; and
d) allowing the organic phase to evaporate and thereby obtain particulate carriers comprising a hollow lumen containing biologically active agents in an aqueous phase.

26. The method of claim 25 wherein the ocular delivery is periocular such as for example is trans-scleral, subconjunctival, sub-tenon, peribulbar, topical, retrobulbar or is delivered to the inferior, superior or lateral rectus muscle.

27. The method of claim 26 wherein the ocular delivery is trans-scleral.

28. The method of any one of claims 25 to 27 wherein the particulate carrier is a microsphere.

29. The method of any one of claims 25 to 28 wherein the biologically active agent is a protein.

30. The method of any one of claims 25 to 29 wherein the biologically active agent is a dAb.

31. The method of any one of claims 25 to 28 wherein the particulate carrier is a microsphere and the biologically active agent is a protein.

32. The method of any one of claims 25 to 30 wherein the particulate carrier is a microsphere and the biologically active agent is a dAb.

33. A method of producing the nanoparticles of any one of claims 1 to 10 comprising the steps of:
a) dissolving a polymer in an organic solvent to form a polymer solution;
b) adding an aqueous solution containing a protein to the polymer solution to form a primary emulsion of aqueous phase droplets in a continuous organic phase;
c) mixing the primary emulsion with an aqueous medium to form a secondary emulsion; and
d) allowing the organic phase to evaporate and thereby obtain nanoparticles comprising a hollow lumen containing proteins in an aqueous phase.

34) The method as claimed in any one of claims 25 to 33 wherein the polymer comprises poly-L-lactide (PLA), poly butylcyanoacrylate (PBCA) or poly(lactide-co-glycolide)(PLG), or poly(caprolactone), poly(hydroxybutyrate) and/or copolymers thereof.

35) The method as claimed in claim 34 wherein the polymer is pegylated.

36) A method of producing a particulate carrier comprising the steps of:
a) dissolving polybutylcyanoacrylate (PBCA) in an organic solvent to form a polymer solution;
b) adding an aqueous solution containing a biologically active agent to the polymer solution to form a primary emulsion of aqueous phase droplets in a continuous organic phase;
c) mixing the primary emulsion with an aqueous medium to form a secondary emulsion; and
d) allowing the organic phase to evaporate and thereby obtain particulate carriers comprising a hollow lumen containing the biologically active agent in an aqueous phase.

37. The method of claim 36 wherein the particulate carrier is a nanoparticle

38. The method of claim 36 or 37 wherein the biologically active agent is a protein.

39. The method of claim 36 wherein the particulate carrier is a nanoparticle and the biologically active agent is a protein.

40. The method of any one of claims 25 to 39 wherein the protein to polymer ratio in the nanoparticle is at least about 5% W/W.

41. The method of any one of claims 25 to 40 wherein step (a) further comprises the addition of a gel forming agent.

42. The method of claim 41 wherein the gel forming agent is agarose.

43. A method of delivering a protein across the blood brain barrier by encapsulating the protein in a nanoparticle and administering the protein to a human or animal in need thereof.

44. A method of delivering a domain antibody for ocular delivery by encapsulating the domain antibody in a microsphere and administering the domain antibody to a human or animal in need thereof.

48. A pharmaceutical composition comprising a protein encapsulated in nanoparticles produced by the methods of any one of claims 13 to 45.

49. A pharmaceutical composition comprising a dAb encapsulated in microspheres produced by the methods of any one of claims 13 to 45.

50. The composition of claim 48 wherein the protein is used to treat disorders or diseases of the central nervous system.

51. The composition of claim 40 wherein the dAb is used to treat ocular diseases.
